(19) United States
(12) Patent Application Publication
(10) Pub. No.: US 2014/0303516 A1
(43) Pub. Date: Oct. 9, 2014

(54) URINE COLLECTION DEVICE

(71) Applicant: Scott Schneider, St. Louis Park, MN (US)

(72) Inventor: Scott Schneider, St. Louis Park, MN (US)

(21) Appl. No.: 13/965,910

(22) Filed: Aug. 13, 2013

Related U.S. Application Data
(60) Provisional application No. 61/809,030, filed on Apr. 5, 2013.

(51) Int. Cl. A61B 10/00

(52) U.S. Cl. CPC A61B 10/007
USPC 600/573

(57) ABSTRACT

A disposable urine collection device is provided for collection of a urine sample from a patient that is uncooperative or unable to provide a voluntary midstream urine specimen. The urine collection device is configured so as to absorb a releasable volume of urine appropriate for urinalysis, without interfering with various parameters of urinalysis, including analyte detection.

### CROSS-REFERENCE TO RELATED APPLICATION

This application claims the benefit of U.S. Provisional Application No. 61/809,030, filed Apr. 5, 2013, which is incorporated herein by reference.

### BACKGROUND OF THE INVENTION

1. Field of the Invention

A disposable urine collection device is provided for collection of a urine sample from a patient that is uncooperative or unable to provide a midstream urine specimen. The urine collection device is configured so as to absorb a releasable volume of urine appropriate for urinalysis, without interfering with various parameters of urinalysis, including analyte detection.

2. Description of the Related Art

The U.S. population is aging. Persons 65 years or older numbered 39.6 million in 2009, and represented about 12.9% of the US population. More than 16 million people in the United States are living with cognitive impairment, and it is estimated that over 5 million Americans age 65 and older may currently have Alzheimer's disease, the most well-known form of cognitive impairment. The average Medicaid nursing facility expenditure per state in 2010 for individuals with Alzheimer's disease is estimated at $647 million, not including home- and community based care or prescription drug costs. The aging of the U.S. population will create challenges for financing nursing home care, assisted living and home health care in the future, particularly for those with cognitive impairment.

Urinalysis is a valuable tool for diagnosis of urologic conditions such as calculi, urinary tract infection (UTI), and malignancy, and can be used to help diagnose systemic disease. Urine is typically sampled by midstream urine collection (MSU), suprapubic puncture, and catheter insertion. Suprapubic aspirate is free of contaminating organisms, but the procedure is invasive and limited to a few specific clinical situations. Urine collection from a bedridden or unconscious patient usually includes a catheter inserted directly into the urethra of the patient to collect urine as it is produced or as micturition occurs. Inherent in catheterization and MSU is possible contamination by perineal, vaginal, fecal, or skin flora. MSU sampling is the most common method but has a high inherent contamination rate defined by mixed growth cultures and growth of non-pathogenic commensals. Many routine urinalyses, however, do not involve microscopy or culture, so MSU, or equivalent urine collection, is acceptable.

One difficulty facing health care personnel is urine sample collection from cognitively impaired and uncooperative patients.

Collection of a routine urine sample for urinalysis can require a significant time commitment, particularly from a cognitively impaired, incontinent or uncooperative patient. The patient may be given extra fluids to drink to encourage urination, but the patient may be uncooperative or incontinent, requiring the health care worker to monitor the patient for an extended period of time.

Inability to collect a voluntary routine urine sample from the patient often results in straight catheterization which is time consuming and uncomfortable for the patient. In addition, straight catheterization is undesirable in that it increases risk for urinary tract infection. Further, Medicare will reimburse only a fixed amount corresponding to catheterization supplies to a medical agency.

Various urine collection devices (UCDs) are known for both elimination and urinalysis. UCDs for urinalysis range from a simple plastic cup, or “Texas hat”, to be used in association with a toilet, to elaborate devices to collect specific volumes or types of urine samples at various points in the urination (micturition) process.

U.S. Pat. No. 4,457,314 issued on Jul. 3, 1984 to Knowles claims a female urine collection device and method which includes a cup-shaped receiving chamber with a flexible lip which seals around the urethral orifice. A duct section leads to flexible tubing leading to a compressible collection reservoir. The chamber and reservoir are mounted in a housing of soft absorbent material.

U.S. Pat. No. 5,678,564 issued on Oct. 21, 1997 to Lawrence et al. describes a liquid removal system which utilizes a membranous interface device which maintains a vacuum on one side. The vacuum operates such that liquid passes through the membrane and is removed to a receptacle for disposal.

U.S. Pat. No. 4,756,029 issued on Jul. 12, 1988 to Zieve et al. describes a feminine urinary device which permits urinating in a standing position. The device has a hingedly attached flap with expandable walls which permit the side walls to expand downward in order to collect the urine. The pad is held in place against the body for the passage of urine into the expandable receptacle. Alternatively, the device can be designed to include an aperture which attaches to a spout which acts as a conduit for the urine.

U.S. Pat. No. 4,781,713 issued on Nov. 1, 1988 to Welch et al. describes a disposable incontinency pad for females which includes an absorbent pad encased in a moisture resistant layer to prevent leakage. The device includes an ovoid opening with a contoured rim portion which conforms to the urinary area of the wearer. The rim directs urine into the pad and away from the user.

U.S. Pat. No. 7,171,699 issued on Feb. 6, 2007 to Ernest et al. describes a disposable urine collection device for use by a person which includes an absorbent pad for the collection of urine and a glove attached to the absorbent pad. The glove design includes a mitt for insertion of the user's hand into the device under the absorbent pad through the sleeve of the glove.

U.S. Patent Application publication No. 20060200102, published Sep. 7, 2006, describes a disposable urinary collection device for a male patient comprising a bulb-shaped sac formed from a single sheet of material. The sheet has edges drawn together by a first elastic member to form a gathered hand insertion opening. An outer perimeter of the passageway has a second elastic member for securing the sac to the male patient.

U.S. Patent Application publication No. US 20070010797, published Jan. 11, 2007, describes multi-functional urine collection devices, embodiments of which can include a self-expanding container having a receptacle for receiving urine from the tubing, a pump for moving urine through the tubing and into a receptacle, extendable tubing that may be shortened and/or lengthened, and/or one or more meters for monitoring, measuring, transmitting or storing a characteristic from the urine.

U.S. Patent Application publication No. 20080274495, published Nov. 6, 2008, provides a diagnostic method for testing hydration and other conditions. Methods for the non-invasive collection of a liquid sample in small quantities, such as urine for testing hydration in infants and incontinent adults, directly at the source of that sample, isolation of the collected sample from a testing site, the transportation of that sample to a site for testing contained within the device itself, and the selective application of some or all of the specimen to a testing apparatus contained within the device itself, with a minimum of specimen handling required. A multi-layered, contoured sample collection component is provided where the engagement surface may be configured to releasably affix to a diaper, and is appropriate for collecting small volumes of urine, for example at least about 20 microliters, or at least about 1 microliter

None of the inventions described above provide an economical, disposable urine collection device for use by a health care worker in a method for collecting a urine sample in a volume sufficient for laboratory urinalysis from a patient that may be uncooperative or unable to provide a midstream urine sample. A simple method for routine urine sample collection is desirable.

A method for collecting a urine sample is provided comprising providing a urine collection device; attaching the device to the external surface of a patient's clothing or permeable undergarment wherein the patient is known to be uncooperative or incontinent; encouraging the patient to provide a routine urine sample; failing the ability to collect a routine urine sample, the device is detached from the patient's clothing or undergarment following micturition and wetting of the device; expressing urine from the device into a sample container; and labeling and submitting the sample container to a laboratory for urinalysis or drug panel testing.

### SUMMARY OF THE INVENTION

A disposable urine collection device is provided for collection of a urine sample from a patient, for example, a patient that is uncooperative or unable to provide a midstream urine specimen. The urine collection device is configured so as to absorb a releasable volume of urine appropriate for urinalysis, without interfering with various parameters of urinalysis, including analyte detection.

In some embodiments, a urine collection device is provided having an internal face to be positioned toward a patient and an external face to be positioned away from the patient, comprising a first layer consisting of an absorbent layer, and a means for temporary attachment to a patient's clothing, wherein the urine collection device is configured so as to cover the urethral area of the patient, but not the anal area of the patient.

In some embodiments, the urine collection device further comprises a fluid impermeable backing sheet covering the external face of the device.

In some embodiments, the urine collection device comprises a means for attachment that is one or more adhesive strips along a border of the internal face, wherein the adhesive strips are covered with a removable cover.

In some embodiments, the urine collection device comprises a permeable facing sheet covering the internal face. In some aspects, the facing sheet is a rayon facing sheet.

In some embodiments, the urine collection device comprises a backing sheet, for example, a polyethylene backing sheet.

In some embodiments, the urine collection device comprises an absorbent layer selected from one or more of porous ceramics, glass fibers, wood fibers, cloth, cellulose, sponge, polyester, polypropylene, and polyamide.

In some embodiments, the urine collection device comprises the absorbent layer is selected from cellulose or polypropylene.

In some embodiments, the urine collection device comprises a two-dimensional surface area greater than 100 square inches and less than 500 square inches. In some embodiments, the UCD two-dimensional surface area is selected from between about 100 square inches to about 450 square inches. In some embodiments, the UCD two-dimensional surface area is selected from between about 100 square inches to about 200 square inches.

In some embodiments, the urine collection device comprises a cellulose absorbent layer; a polyethylene impermeable backing sheet; a rayon facing sheet; two adhesive strips at opposing borders of the internal face; and an internal face having a two dimensional surface area greater than 100 square inches and less than 500 square inches. In some aspects, the two dimensional surface area of the device is selected from hourglass, diamond, crescent, square, rectangular, trapezoidal, pentagonal, hexagonal, octagonal, oval, circular or triangular in shape. In some aspects, the two dimensional surface area is square or rectangular in shape. In some embodiments, the urine collection device is capable of releasably absorbing at least 20 mL, at least 30 mL, or at least 40 mL of urine.

### BRIEF DESCRIPTION OF THE DRAWINGS

FIG. 1 shows one embodiment of the invention. A urinary collection device 10 is provided comprising an absorbent layer 20, adhesive strip 30 on the internal face, permeable topsheet 40 at the internal face, impermeable backing sheet 50 at the external face, and removable covers 60 to be removed from the adhesive strips 30 at the internal face prior to temporary attachment to clothing or permeable undergarments of the patient.

### DETAILED DESCRIPTION OF THE INVENTION

A disposable urine collection device is provided for collection of a urine sample from a patient that is uncooperative or unable to provide a midstream urine specimen. The urine collection device is configured so as to absorb a releasable volume of urine appropriate for urinalysis, without interfering with various parameters of urinalysis, including analyte detection.

### DEFINITIONS

The term “about” refers to +/−10% of the unit value provided.

The term “accuracy” refers to the ability to get the correct, or true, result.

The term “analyte” refers to the substance to be measured.

The term “blind sample” refers to a control material submitted to the analyst, as an unknown, but analyzed as a routine sample.

Random urine collection can occur at any time of day with no precautions with respect to contamination. The sample may be dilute, isotonic, or hypertonic and may contain white blood cells, bacteria and squamous epithelium. In some embodiments, the urine collection device (UCD) presented herein is useful for collection of a random urine sample. Early morning collection of a sample before fluid ingestion usually results in hypertonic urine exceeding specific gravity of 1.022 in a healthy individual. In some embodiments, the UCD herein is useful for early morning urine collection.

Complete urinalysis includes physical, chemical and microscopic examinations. Urinalysis (UA), or routine and microscopy (R&M), is a series of tests performed on urine and is one of the most common methods of medical diagnosis. Many analytes, cells, and properties of urine are routinely analyzed, including specific gravity, and description of color and appearance. Target parameters include ions and trace minerals such as nitrite, fractional excretion of sodium, potassium, urinary calcium, and phosphate. Other target parameters include proteins (proteinuria), enzymes such as human chorionic gonadotropin (hCG), blood cells (erythrocytes, (red blood cells, RBCs), leukocytes (white blood cells, WBCs), and hemoglobin (“blood”)). Other molecules routinely measured in urinalysis include glucose, ketone bodies, bilirubin, urobilinogen, creatinine, catecholamines, dopamine, free cortisol, and phenylalanine.

The two key markers for chronic kidney disease are estimated glomerular filtration rate and urine albumin. Urine albumin is a key marker for chronic kidney disease. Urinary albumin to urinary creatinine ratio (UACR or UA/UC) is assessed to determine albumin excretion in mg/day. Albuminuria is present when UACR is greater than 30 mg/g and is a marker for CKD. More frequent monitoring may be indicated with patients with changing clinical status, for example, in patients with type 1 or type 2 diabetes, or after therapeutic interventions.

Other urine parameters include urine specific gravity, osmolality, pH, and bacterial cultures. Urine can also be analyzed to determine the presence of therapeutic drugs, metabolites and drugs of abuse. A routine urinalysis does not include microscopy or culture.

Some tests in urinalysis are performed using urine test strips. Various urine test strips can be used to quantify leukocytes (leukocyturia), nitrite (nitrituria), protein (proteinuria, albuminuria, microalbuminuria), blood (hematuria), and specific gravity.

Microscopic examination of urine is performed to determine numbers and types of cells and material such as urinary casts aid in various specific diagnosis or prognosis in the patient. Cast formation can occur under conditions of low flow rate, high salt, concentration, low pH each of which helps cause protein precipitation. Urinary casts are formed in the distal convoluted tubule, or the collecting duct. Hyaline casts are formed primarily of mucoproteins, but can sometimes be seen in healthy patients. Red blood cell casts are indicative of glomerulonephritis. White blood cell casts are typical for acute pyelonephritis or sometime glomerulonephritis, and indicate inflammation in the kidney.

Some crystals can form even in healthy patients are usually composed of calcium oxalate, triple phosphate crystals and amorphous phosphates. Cystine, tyrosine or leucine crystals, although uncommon, can form in cases of severe kidney disease.

Cloudy urine can result from precipitated phosphate crystals (phosphaturia) in alkaline urine, but pyuria can also be the cause. Cloudy urine can also be caused by chyluria, lipiduria, hyperoxaluria and diets high in purine-rich foods (hyperuricosuria).

Macroscopic analysis includes direct visual observation. Normal fresh urine is pale to dark yellow or amber in color and clear. Normal volume of urine is 750 to 2000 mL per 24 hour period. Cloudiness or turbidity can be caused by excessive protein or cellular material in the urine, or may develop from crystallization or precipitation of salts upon standing or under refrigeration. If the specimen clears after addition of a small amount of acid, then precipitation of salts is the probable cause of turbidity. A red or reddish-brown color may result from eating fresh beets, a food dye, a drug or the presence of myoglobin or hemoglobin in the urine. A cloudy red specimen could be caused by the presence of many red blood cells. In some embodiments, the result of macroscopic analysis of an expressed urine sample from the urinary collection device pad is the same as that of the urine prior to being absorbed by the pad.

Urinary pH may range from pH 4.5 to pH 8.0 with an average normal range of pH 5.5 to 6.5. In some embodiments, a reference range is pH 5.0-8.0.

Specific gravity of urine measures the urine density, or the ability of the kidney to concentrate or dilute the urine over that of plasma. Dipsticks are available to measure specific gravity; however, most labs measure specific gravity with a refractometer. Specific gravity between 1.002 and 1.035 is considered normal for a random sample in a patient with normal kidney function. The specific gravity of the glomerular filtrate in Bowman's space ranges from 1.007 to 1.010, a measurement below this range is indicative of hydration, or over this range is indicative of dehydration. If specific gravity is not over 1.022 after 12 hours without food or water, renal concentrating ability is impaired; possibly due to renal impairment or nephrogenic diabetes insipidus. In end-stage renal disease specific gravity becomes 1.007 to 1.010. A urine specific gravity over 1.035 may be contaminated, contain high levels of glucose or the patient may have recently received high density radiopaque dyes, or low molecular weight dextran solutions.

Protein can be screened by dipstick screening, or semi-quantitative, or quantitative tests. Dipstick bromphenol blue detects albumin, and also globulin, but poorly detects some proteins such as Bence-Jones proteins. Precipitation by heat is used in semi-quantitative tests, but sulfosalicylic acid method is more sensitive precipitation test that can detect albumin, globulin and Bence-Jones proteins at low concentrations. Normally only small proteins are filtered/reabsorbed by renal tubule. Normal protein excretion does not exceed 150 mg/24 hours or 10 mg/100 mL in a single specimen. More than 150 mg/day is defined as proteinuria. More than 3.5 g in 24 hours is severe and known as nephrotic syndrome.

Glucose can be detected by dipstick, for example, by glucose oxidase reaction; or by other tests, such as Benedicts copper reduction test. Typically less than 0.1% of glucose filtered by the glomerulus appears in urine, or less than 130 mg/24 hours. Glycosuria, or excess glucose in the urine, is indicative of diabetes mellitis.

Ketones resulting from diabetic ketosis or some other form of calorie deprivation can be detected by dipstick or test tablets containing sodium nitroprusside. Ketones in the urine, or ketone bodies, include, for example, acetone, acetoacetic acid, and beta-hydroxybutyric acid.

Leukocyte esterase positive test results can occur from the presence of white blood cells in whole or lysed cells. Pyuria can be detected even if the sample has damaged or lysed WBCs. A negative leukocyte esterase test means that infection is not likely and without additional evidence of urinary tract infection, microscopic examination and or urine culture need not be performed to rule out significant bacteriuria.

Red blood cells in urine can be indicative of a number of abnormal conditions, for example, glomerular damage, kidney trauma, urinary tract infections, nephrotoxins, renal infarcts, urinary tract stones, acute tubular necrosis, erosion of urinary tract tumors, or physical stress. Red blood cells can also appear in samples from contamination, or trauma produced by catheterization. Red blood cells may appear swollen by dilute urine or crenated by concentrated urine. Dysmorphic red blood cells may suggest a glomerular disease such as glomerulonephritis.

Nitrite test positive samples indicate that bacteria may be present in significant numbers. Gram negative rods such as E. coli are more likely to give a positive test.

Analysis of various metabolites by urinalysis is known. For example, methacetin can be used to evaluate hepatic metabolism. For example, a [¹⁵N]methacetin urine test can be used to study human 0-demethylase activity to characterize hepatic detoxification capacity following oral administration of an aqueous solution. (Krumbiegel at al., [¹⁵N]Methacetin urine test: A method to study the development of hepatic detoxification capacity, 1990, European J. Pediatrics, 149(6); 393-395).

Urine testing for various drugs of abuse is well known. Marijuana/cannabinoid; cocaine, amphetamine; methamphetamine; opiates; phencyclidine; alcohol; lysergic acid diethylamide; methaqualone; barbiturates; and benzodiazepines are routine analytes in urinalysis.

Urine testing for therapeutic drug monitoring is also useful, for example, for monitoring opiates. Urine testing for various drugs can be performed, for example, by using immunoassay or ELISA, for example, screens for alcohol, amphetamines, barbiturates, benzodiazepines, cannabinoids, cocaine, fentanyl, methadone, opiates, phencyclidine or propoxyphene is common.

A urinary collection device (UCD), comprising an absorbent pad and means for attachment of the UCD to a patients clothing is provided. The urine collection device is used to collect urine from a patient unwilling or unable to provide a urine specimen without catheterization. The urine collection device provides a urine sample appropriate for urinalysis or UA/UC.

In some embodiments, the UCD comprises an absorbent pad, of such dimensions to allow collection of 2 mL or more, 5 mL or more, 10 mL or more, 15 mL or more, 20 mL or more, 25 mL or more, or 30 mL or more volume of urine for urinalysis. In some embodiments, the dimensions of the absorbent material should allow the UCD to releasably absorb at least 30 mL, at least 40 mL, at least 50 mL, or at least 60 mL of urine from the patient. In some embodiments, the absorbent material in the UCD device can absorb and release at least 30 mL of urine from the patient. In some embodiments, the absorbent layer can releasably absorb from 5 mL to 200 mL, 10 mL to 150 mL, 30 mL to 100 mL, or 40 mL to 60 mL of urine from the patient.

The absorbent material in the present UCD will absorb the appropriate volume of urine and release at least 60% or more, 70% or more, 75% or more, 80% or more, 85% or more, 90% or more or 95% or more of the volume of urine collected.

A method for urine sample collection is provided. The UCD is attached to the clothing of the patient. In some embodiments, the UCD is attached to the external portion of the patients clothing over the urogenital area. In some embodiments, the UCD is attached between layers of the patients clothing. In some embodiments, the UCD is attached to an internal portion of the patients clothing. In some embodiments, the UCD is attached to the urogenital side of a “Depends” or other adult diaper. If the present UCD is attached internally, or adjacent to the patient's skin, the UCD shall not cover the anal area of the patient.

A method for collecting a urine sample comprising using the urinary collection device is provided. A random urine sample is collected from the patient at any time of day, or in the morning prior to consumption of any liquids. In some embodiments, the patient is given extra fluids to drink to encourage urination. Optimally, the patient will be cooperative, allowing for collection of urine in a plastic cup, or “Texas hat” urinary collection device, such as that fitted to a toilet. However, if the patient is not cooperative, or the patient is incontinent, micturition occurs through the patients clothing and the urine is absorbed by the collection pad of the UCD. Following the UCD is removed from the patient's clothing and urine is expressed from the absorbent pad by squeezing into a container such as a sample collection cup or tube. Alternatively, the UCD is removed from the patient's clothing and placed in a container for later expression, such as a plastic, metal, or glass container or plastic bag. The filled container is labeled and transported to a laboratory for urinalysis. Alternatively, the container is labeled and the urinalysis is performed on site. Alternatively, the container is pre-labeled prior to adding the expressed sample or un-expressed UCD.

In some embodiments, the UCD includes a single liquid absorbent material present in one or more, two or more, or three or more layers. In some embodiments, the UCD includes two or more different liquid absorbent materials present in two or more, or three or more layers. In some embodiments, the UCD has two or more layers of different materials, where a first layer is a moisture impermeable layer and a second layer is a liquid absorbent layer. In some embodiments, the UCD has three or more layers of different materials, where a first layer is a moisture impermeable layer, a second layer is a liquid absorbent layer, and a third layer is a moisture permeable topsheet.

Top Sheet

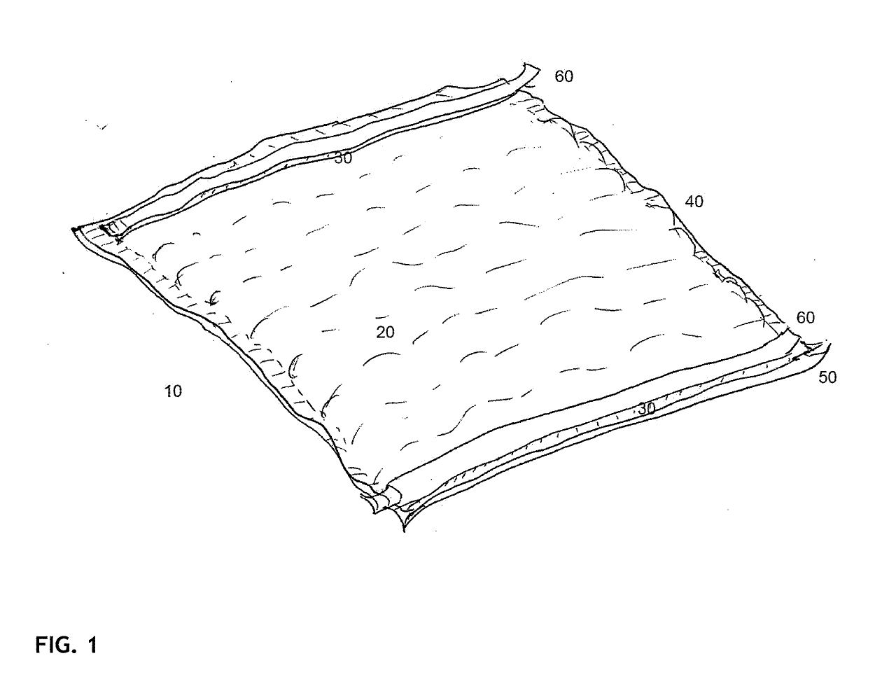

In some embodiments, the UCD has an absorbent layer and a moisture-permeable top sheet, or facing sheet, 40, on the internal face, as shown in FIG. 1. In some embodiments, the facing sheet is a rayon facing sheet. In some embodiments, the optional top sheet helps hold the absorbent layer in place during expression of a urine sample from the wetted UCD. Alternatively, the optional top sheet can be removed, or partially removed, prior to expression of the urine sample from the UCD to decrease contamination, should the UCD be worn adjacent to the patient's skin.

Absorbent Material

The absorbent material 20, as shown in FIG. 1, may be selected from, for example, one or a combination of hydrophilic materials such as porous ceramics, glass fibers, wood fibers, cloth, cellulose, sponge, polyester, polypropylene, and polyamide matrices. Optimally, the absorbent material will exhibit low protein binding to enable accuracy of urinalysis and minimal loss of target analyte. In some embodiments, the absorbent material is selected from polypropylene or cellulose. In some embodiments, the absorbent pad is a cellulose pad with a biodegradable polyethylene backing. The absorbent material is selected such that minimal contamination, or change in appearance, chemistry or analyte profile occurs upon expression of a urine sample from the wetted UCD. In some embodiments, the absorbent material is an absorbent polypropylene material. The absorbent material may be present in one or more, two or more or three or more homogeneous or heterogeneous layers.

In some embodiments, the absorbent material is ethylene oxide and or propylene oxide treated to reduce protein binding as disclosed in WO2000074740, which is incorporated herein by reference.

The dimensions of the absorbent material should allow the UCD to releasably absorb at least 30 mL, at least 40 mL, at least 50 mL, or at least 60 mL of urine form the patient. In some embodiments, the absorbent material in the UCD device can absorb and release at least 30 mL of urine from the patient. In some embodiments, the absorbent layer can releasably absorb from 5 mL to 200 mL, 10 mL to 150 mL, 30 mL to 100 mL, or 40 mL to 60 mL of urine from the patient. In some embodiments, the absorbent layer is configured so as to releasably absorb at least 10 mL, at least 20 mL, at least 30 mL, at least 40 mL, or at least 50 mL of urine from the patient.

In some embodiments, the UCD comprises an absorbent material pad in a 12 inch by 12 inch square. In some embodiments, the UCD comprises an absorbent material pad in a 12 inch by 11.5 inch rectangle. In some embodiments, the UCD comprises an absorbent material pad in a 17 inch by 24 inch rectangle.

The urinary collection device has an internal face, to be worn toward the patient; and an external face, to be worn away from the patient.

In some embodiments, the UCD comprises an absorbent layer and a means for attachment, having one or more, two or more, three or more layers, provided in a two dimensional shape having a surface area. The two-dimensional shape is selected from a polygon, for example, a square, rectangle, or triangle; or diamond, oval, hourglass, heart, circular or contoured shape such that the two-dimensional surface area is greater than about 100 square inches, greater than about 120 square inches, greater than about 130 square inches, greater than about 150 square inches, or greater than about 200 square inches, or greater than about 300 square inches. In some embodiments, the surface area of the UCD is from about 100 square inches to about 500 square inches. In some embodiments, the surface area of the UCD is from about 130 square inches to about 450 square inches. In some embodiments, the surface area of the UCD is from about 130 square inches to about 150 square inches. In any case, the shape and dimensions of the UCD should cover the urethral area of the patient, but not the anal area of the patient.

In some embodiments, the material of the absorbent layer is selected such that a healthy normal urine sample will not be contaminated or exhibit an abnormal urinalysis test result. In some embodiments, the material of the absorbent pad is selected such that urinalysis results for an abnormal sample expressed from the UCD will reflect urinalysis results of a non-absorbed urine sample collected from the same patient collected at the same time. In some embodiments, the material of the absorbent pad is selected such that urinalysis

Impermeable Backing

In some embodiments, the absorbent material has a moisture impermeable or moisture-resistant backing sheet, 50, as shown in FIG. 1. In some embodiments, the backing sheet is selected from a polyethylene backing, thin plastic, or a natural fiber backing material. In some embodiments, the absorbent pad of the UCD has a biodegradable, moisture-resistant backing sheet that will not interfere with urinalysis results of a sample expressed from the UCD. The optional backing sheet allows for better retention of the urine sample, less contamination of the urine sample, and increased ease of handling of the wetted UCD by a health care worker.

Means for Attachment

The urinary collection device comprises an absorbent pad and means for attaching the absorbent pad to a patients clothing, or permeable undergarments. Referring to FIG. 1, in some embodiments, the means for securing is one or more adhesive surfaces 30, such as a pressure-sensitive adhesive strip, or a double-sided, self-adhesive strip, either of which is normally covered by a removable cover. In some embodiments, the one or more double-sided, self-adhesive strips are selected from commercially-available pressure-sensitive double-sided tape. In some embodiments, the pressure-sensitive adhesive strip is formed by applying a rubber-based hot melt adhesive or the like. The adhesive strip is exposed by removing the removable cover 60 to temporarily attach the UCD to a permeable garment when subjected to pressure.

The adhesive strips are positioned, for example, at intervals at the corners of a polygon shaped device, or along two or more internal face edges, or at two or more regular intervals along the internal face of the UCD, for example, as shown in FIG. 1 at 30.

In some embodiments, the adhesive strip is provided on the internal, absorbent face of the urine collection device in order to temporarily fasten the internal, absorbent face of the UCD to the outside surface of the patient's permeable clothing or permeable undergarments.

In another embodiment, a UCD is provided in a different configuration, with means of attachment on the external face of the UCD to allow temporary attachment to a “Depends”, or diaper-type garment; the means for attachment is positioned on the external face of the UCD, away from the patient, and the UCD is positioned such that the urethral area, but not the anal area, of the patient is covered by the UCD.

### EXAMPLES

### Example 1

### Collection of Urine, Urinalysis and Drug Test Panel

Three absorbent materials were tested for use in the urinary collection device (UCD) and evaluated based upon the ability to absorb a urine sample, retain urine and release a urine sample, appropriate for urinalysis without significant contamination. A large volume urine sample was collected from a healthy 30 yr. old volunteer and divided into four containers. Three absorbent materials were used to absorb the split urine sample, and test urine samples were expressed by manually pressing the wetted absorbent material into individual sample cups. The three test samples and one control sample were submitted as blind samples and urinalysis and drug testing panels were run by a commercial laboratory.

Test sample 1 absorbent material was polypropylene, W×L, 12″×12″ (Sigma-Aldrich, SPILFYTER® hazmat spill control Product Z201057). Test sample 2 absorbent material was cellulose W×L 11.5″×12″ (NPS Corp., SPILFYTER® cellulose-based cellulose sorbent pad, Product 151012). Test sample 3 absorbent pad was fluffed cellulose with polyethylene backing 6 mil, and spun bond nonwoven rayon facing sheet, W×L, 17″×24″ (Labsource Inc., bench underpads, Product L56-145). A minimum of 30 mL urine sample was required by the lab to perform the tests.

Results

Results of urinalysis are shown in Table 1. In general, a microscopic examination of each of the test and the control samples was not indicated due to normal chemical analysis. In general, the laboratory report indicated that clear, cloudy and turbid urines have not shown pathologic findings when the chemical analysis of the urine is normal. As shown in Table 1, no change in the color of the test samples was noted compared to the control sample. The appearance of the control sample was clear; two of the test samples were turbid, and one of the test samples was clear. No significant change in the glucose, bilirubin, ketone, protein, urobilinogen, nitrite, blood, or leukocyte esterase values was observed between any of the three test samples and the control sample. The pH of test sample 1, JJ, was slightly higher than the control sample. The specific gravity of test sample 1, JJ, was slightly higher than the control sample. However, both pH and specific gravity for test sample 1 were well within the normal range and not significantly different than the control sample.

The urinalysis test results indicated that any of the three test absorbent materials would be appropriate for use in a UCD for urine sample collection and urinalysis. In some embodiments, a UCD is provided having a polypropylene absorbent material. In some embodiments, a UCD is provided having a cellulose, or cellulose-based, absorbent material. In some embodiments, a UCD is provided having an absorbent material and a rayon facing sheet. In some embodiments, a UCD is provided having an absorbent material and polyethylene backing sheet. In some embodiments, a UCD is provided having a cellulose absorbent material, a rayon facing sheet, and a polyethylene backing sheet.

TABLE 1
Urinalysis Test Results.

| | Reference | Control | Test 1 | Test 2 | Test 3 |
|---|---|---|---|---|---|
| Test | Range | SS | JJ | JD | JS |
| Color | YELW, | YELLOW | YELLOW | YELLOW | YELLOW |
| | DK. YELW | | | | |
| Appearance | NEGATIVE | CLEAR | TURBID | TURBID | CLEAR |
| Glucose | NEGATIVE | 1 + (H) | 1 + (H) | 1 + (H) | 1 + (H) |
| Bilirubin | NEGATIVE | NEGATIVE | NEGATIVE | NEGATIVE | NEGATIVE |
| Ketone | NEGATIVE | NEGATIVE | NEGATIVE | NEGATIVE | NEGATIVE |
| Specific | 1.001-1.030 | 1.016 | 1.017 | 1.016 | 1.016 |
| Gravity | | | | | |
| pH | 5.0-8.0 | 5.5 | 6.0 | 5.5 | 5.5 |
| Protein | NEG or | NEGATIVE | NEGATIVE | NEGATIVE | NEGATIVE |
| | TRACE | | | | |
| Urobilinogen | 0-1.9 mg/dL | <2.0 | <2.0 | <2.0 | <2.0 |
| Nitrite | NEGATIVE | NEGATIVE | NEGATIVE | NEGATIVE | NEGATIVE |
| Blood | NEGATIVE | NEGATIVE | NEGATIVE | NEGATIVE | NEGATIVE |
| Leukocyte | NEGATIVE | NEGATIVE | NEGATIVE | NEGATIVE | NEGATIVE |
| esterase | | | | | |

Results of urinalysis test indicates that test results for each of the three test samples did not significantly vary from test results from the control sample with respect to color, glucose, bilirubin, ketone, specific gravity, pH, protein, urobilinogen, nitrite, blood, or leukocyte esterase. No false positive test result indicates that any of the three test absorbent materials would be appropriate for use in a UCD for urine sample collection and urinalysis.

Drug Test Panel.

The control and three test samples were also run in a Drug Test Panel, 10 drug OPIEX via GC/MS, and/or LC-MS/MS, for the following drugs and drug classes at the indicated threshold cut-off levels as indicated in Table 2.

TABLE 2
Drug Panel Test Results.

| | | Confi- | | | | |
|---|---|---|---|---|---|---|
| | Screening | rmation | | | | |
| | Level | Level | Con- | Test | Test | Test |
| Description | (ng/mL) | (ng/mL) | trol | 1 | 2 | 3 |
| Amphetamines | 1000 | 500 | NEG | NEG | NEG | NEG |
| Barbiturates | 300 | 200 | NEG | NEG | NEG | NEG |
| Benzodiazepines | 300 | 300 | NEG | NEG | NEG | NEG |
| Benzoylecgonine- | 300 | 150 | NEG | NEG | NEG | NEG |
| cocaine metab | | | | | | |
| Extended opiates | 300 | 300 | NEG | NEG | NEG | NEG |
| Marijuana | 50 | 15 | NEG | NEG | NEG | NEG |
| metabolite | | | | | | |
| Methadone | 300 | 300 | NEG | NEG | NEG | NEG |
| Methylenedioxy- | 500 | 300 | NEG | NEG | NEG | NEG |
| methamphetamine | | | | | | |
| Phencyclidene | 25 | 25 | NEG | NEG | NEG | NEG |
| Propoxyphene | 300 | 300 | NEG | NEG | NEG | NEG |

Results.

The control sample and each of the three test samples were negative for each drug of the panel. No false positive test results is indicative that any of the three test absorbent materials would be appropriate for use in a UCD for urine sample collection and analysis in a drug panel test.

### REFERENCES CITED

Carver, M. C., Jessie, A., (May 31, 2011) “Patient-Centered Care in a Medical Home” OJIN: The Online Journal of Issues in Nursing Vol. 16, No. 2, Manuscript 4.
U.S. Dept. of Health & Human Services, Administration on Aging. Aging Statistics. www.AoA.gov/.
http://www.cdc.gov/aging/pdf/cognitive_impairment/cogImp_policy_final.pdf
National Institute on Drug Abuse (NIDA) Research Monograph 73, 1986, Urine Testing for Drugs of Abuse. Hawks, Chiang, Eds.
U.S. Pat. No. 4,457,314
U.S. Pat. No. 5,678,564
U.S. Pat. No. 4,756,029
U.S. Pat. No. 4,781,713
U.S. Pat. No. 7,171,699
U.S. Patent Application publication No. 20060200102
U.S. Patent Application publication No. 20070010797
U.S. Patent Application publication No. 20080274495
WO 2000/074740

We claim:

1. A urine collection device (UCD) having an internal face to be positioned toward a patient and an external face to be positioned away from the patient, comprising
a first layer consisting of an absorbent layer; and
a means for temporary attachment to a patient's clothing,
wherein the urine collection device is configured so as to cover the urethral area of the patient, but not the anal area of the patient.

2. The urine collection device of claim 1, further comprising
a fluid impermeable backing sheet covering the external face of the device.

3. The urine collection device of claim 1 wherein the means for attachment is one or more adhesive strips at a border of the internal face.

4. The urine collection device of claim 3, wherein the adhesive strips are covered with a removable cover.

5. The urine collection device of claim 1 further comprising a permeable facing sheet covering the internal face.

6. The urine collection device of claim 2, wherein the backing sheet is a polyethylene backing sheet.

7. The urine collection device of claim 5, wherein the facing sheet is a rayon facing sheet.

8. The urinary collection device of claim 1 wherein the absorbent layer is selected from porous ceramics, glass fibers, wood fibers, cloth, cellulose, sponge, polyester, polypropylene, and polyamide.

9. The urinary collection device of claim 8 wherein the absorbent layer is selected from cellulose or polypropylene.

10. The urinary collection device of claim 1 comprising a two-dimensional surface area greater than 100 square inches and less than 500 square inches.

11. The urinary collection device of claim 1 comprising
a cellulose and/or polypropylene absorbent layer;
a polyethylene impermeable backing sheet at the external face;
a rayon facing sheet at the internal face;
two adhesive strips at opposing borders of the internal face; and
a square or rectangular internal face having a two dimensional surface area greater than 100 square inches and less than 500 square inches.

12. The urine collection device of claim 11, wherein the UCD device can absorb and release at least 20 mL of urine from the patient.

13. The urine collection device of claim 11, wherein the UCD device can absorb and release at least 30 mL of urine from the patient.

14. The urine collection device of claim 11, wherein the UCD device can absorb and release at least 40 mL of urine from the patient.
